(19) United States
(12) Patent Application Publication
(10) Pub. No.: US 2021/0137411 A1
(43) Pub. Date: May 13, 2021

(54) SUPPORT DEVICE FOR WEARING DURING A MAGNETIC RESONANCE IMAGING PROCEDURE

(71) Applicant: Nicola A. Hanna, San Diego, CA (US)

(72) Inventor: Nicola A. Hanna, San Diego, CA (US)

(21) Appl. No.: 16/679,812

(22) Filed: Nov. 11, 2019

(51) Int. Cl. A61B 5/055; A41D 13/12

(52) U.S. Cl. CPC A61B 5/0555; A41D 2300/324; A41D 2300/32; A41D 13/1245

(57) ABSTRACT

A support device for wearing during a MRI procedure includes a front panel, a left arm sleeve extending from the left edge of the front panel, and a right arm sleeve extending from the right edge of the front panel. Methods for preparing the support device for wearing during a MRI procedure include attaching the front panel to the left arm sleeve; and attaching the front panel to the right arm sleeve.

### FIELD OF THE INVENTION

The present invention generally relates to a device for supporting the arms of a patient, and more particularly, to a device for supporting the arms of a patient undergoing a medical procedure while in the supine position such as during a magnetic resonance imaging (MRI) procedure.

### BACKGROUND OF THE INVENTION

MRI is a medical imaging technique used in radiology to form pictures of the anatomy and the physiological processes of the body. MRI scanners use strong magnetic fields, magnetic field gradients, and radio waves to generate images of the organs in the body. MRI does not involve X-rays or the use of ionizing radiation, which distinguishes it from computerized tomography (CT) or computerized axial tomography (CAT) scans and positron-emission tomography (PET) scans. MRI is a medical application of nuclear magnetic resonance (NMR).

While the hazards of X-rays are now well controlled in most medical contexts, a MRI scan may still be seen as a better choice than a CT scan. MRI is widely used in hospitals and clinics for medical diagnosis, staging of disease and follow-up without exposing the body to radiation. An MRI may yield different information compared with CT. However, patient discomfort may be associated with MRI scans. Compared with CT scans, MRI scans typically take longer, are louder, and they usually need the subject patient to enter a narrow, confining tube causing body and arm discomfort as well as claustrophobic mental discomfort from being enclosed in a small space for extended periods of time. Therefore, there remains a need in the art for new devices that address the comfort needs of a patient undergoing a medical procedure.

### SUMMARY OF THE INVENTION

The present invention provides a support device that can be worn by a patient when undergoing a medical procedure in the supine position, i.e. on their back, such as required when undergoing MRI, CT or CAT procedures. The body position and prolong time required for these procedures can be very uncomfortable for a patient. The level of discomfort usually increases for patients who are claustrophobic or have higher body mass. When worn by the patient, the support device acts to restrain and/or support the patients arms by their sides of their body, which results in relieving stress and/or discomfort to their arms and shoulders.

Thus, in one embodiment the disclosure provides a support device for wearing during a MRI procedure, which includes a front panel having a bottom edge, a top edge, a left edge, a right edge, a front surface and a rear surface; a left arm sleeve having a bottom edge, a top edge, a front surface and a rear surface, the left arm sleeve extending from the left edge of the front panel; and a right arm sleeve having a bottom edge, a top edge, a front surface and a rear surface, the right arm sleeve extending from the right edge of the front panel.

In another embodiment, the disclosure provides a method for preparing a support device for wearing during a MRI procedure, which includes attaching a front panel having a bottom edge, a top edge, a left edge, a right edge, a front surface and a rear surface to a left arm sleeve having a bottom edge, a top edge, a front surface and a rear surface, the left arm sleeve extending from the left edge of the front panel; and attaching the front panel having the bottom edge, the top edge, the left edge, the right edge, the front surface and the rear surface to a right arm sleeve having a bottom edge, a top edge, a front surface and a rear surface, the right arm sleeve extending from the right edge of the front panel.

### BRIEF DESCRIPTION OF THE DRAWINGS

An understanding of the present invention may be obtained by reference to the accompanying drawings, when considered in conjunction with the subsequent, detailed description, in which:

FIG. 1 illustrates an embodiment of a front view of the support device;

FIG. 2 illustrates an embodiment of a rear view of the support device;

FIGS. 3 and 4 illustrate an embodiment of a right side view and left side view of the support device, respectively;

FIG. 5 illustrates an embodiment of a front view of the support device 100 when being worn by a user, and

FIGS. 6 and 7 illustrate an embodiment of a right side view and left side view of the support device, respectively, when being worn by a user.

For purposes of clarity and brevity, like elements and components will bear the same designations and numbering throughout the Figures.

### DETAILED DESCRIPTION OF THE EMBODIMENTS

The present invention provides a support device that can be worn by a patient when undergoing a medical procedure in the supine position, i.e. on their back, such as required when undergoing MRI or CT procedures. The body position and prolong time required for these procedures can be very uncomfortable for a patient. The level of discomfort usually increases for patients who are claustrophobic or have higher body mass. When worn by the patient, the support device acts to restrain and/or support the patients arms by their sides of their body, which results in relieving stress and/or discomfort to their arms and shoulders.

As designed, the support device allows a patient's arms to be supported during the medical procedure alleviating most discomfort. Each arm can be slotted through an armband holster or sleeve which can be circular or oval in shape. The arm bonds can be connected by a front piece or connector that lays across the patient's chest. The result is a wearable support device that stabilizes a patient's arms in a very comfortable position during the typical 35-45 minute procedure. The appropriate size of the device depends on the size of the user and can be provided in children, women and men sizes ranging in all sizes from small, medium, large, extra large, etc.

FIG. 1 illustrates an embodiment of a front view of the support device 100. As shown in this figure, the support device 100 includes a front panel 101 having a bottom edge 102, a top edge 103, a left edge 104, a right edge 105, a front surface 106 and a rear surface 107 (not shown). The support device 100 further includes a left arm sleeve 108 having a bottom edge opening 109, a top edge opening 110, a front surface 111 and a rear surface 112 (not shown), wherein the left arm sleeve 108 extends from the left edge 104 of the front panel 101; and a right arm sleeve 113 having a bottom edge opening 114, a top edge opening 115, a front surface 116 and a rear surface 117 (not shown), wherein the right arm sleeve 113 extends from the right edge 105 of the front panel 101 (Note: all directional views are from the standpoint of a user wearing the device).

FIG. 2 illustrates an embodiment of a rear view of the support device 100. As shown in this figure, the support device 100 includes the front panel 101 having a bottom edge 102, a top edge 103, a left edge 104, a right edge 105, a front surface 106 (not shown) and a mar surface 107. The support device 100 further includes a left arm sleeve 108 having a bottom edge opening 109, a top edge opening 110, a front surface 111 (not shown) and a rear surface 112; and a right arm sleeve 113 having a bottom edge opening 114, a top edge opening 115, a front surface 116 (not shown) and a rear surface 117.

FIGS. 3 and 4 illustrate an embodiment of a right side view and left side view of the support device 100, respectively. As shown in FIG. 3, the support device 100 includes a front panel 101 having a bottom edge 102, a top edge 103, a right edge 105, a front surface 106; and a right arm sleeve 113 having a bottom edge opening 114, a top edge opening 115, and a front surface 116. As shown in FIG. 4, the support device 100 includes a front panel 101 having a bottom edge 102, a top edge 103, a left edge 104, a front surface 106; and a left arm sleeve 108 having a bottom edge opening 109, a top edge opening 110, and a front surface 111.

FIG. 5 illustrates an embodiment of a front view of the support device 100 when being worn by a user. As shown in this figure, the support device 100 includes a front panel 101 having a bottom edge 102, a top edge 103, a left edge 104, a right edge 105, a front surface 106. The support device 100 further includes a left arm sleeve 108 having a bottom edge opening 109, a top edge opening 110, a front surface 111, wherein the left arm sleeve 108 extends from the left edge 104 of the front panel 101; and a right arm sleeve 113 having a bottom edge opening 114, a top edge opening 115, a front surface 116, wherein the right arm sleeve 113 extends from the right edge 105 of the front panel 101

FIGS. 6 and 7 illustrate an embodiment of a right side view and left side view of the support device, respectively, when being worn by a user. As shown in FIG. 6, the support device 100 includes a front panel 101 having a bottom edge 102, a top edge 103, a right edge 105, a front surface 106; and a right arm sleeve 113 having a bottom edge opening 114, a top edge opening 115, and a front surface 116. As shown in FIG. 7, the support device 100 includes a front panel 101 having a bottom edge 102, a top edge 103, a left edge 104, a front surface 106; and a left arm sleeve 108 having a bottom edge opening 109, a top edge opening 110, and a front surface 111.

In an embodiment, the support device can include an attachment means for attaching the left arm sleeve to the front panel; and an attachment means for attaching the right arm sleeve to the front panel.

In some embodiments, the attachment means includes at least one zipper, at least one button, at least one snap, and/or at least one hook and loop fastener. In some embodiments the attachment means may be between each sleeve and the front panel of the support device. In other embodiments, the front panel may further include another attachment means for joining the front panel together along with the two sleeves.

In other embodiments, the attachment means for attaching the left arm sleeve to the front panel is a hook and loop fastener; and the attachment means for attaching the right arm sleeve to the front panel is a hook and loop fastener.

In some embodiments, the support device includes at least one adjustable strap for securing the left arm sleeve and/or the right arm sleeve to the front panel of the device.

In other embodiments, the support device may be made of one or more pieces of material selected from one or more synthetic materials such as a polyester material, one or more cotton materials, one or more cellulosic/viscose materials, one or more wool materials, one or more silk materials, one or more leather materials, one or more bast fiber materials, and/or one or paper materials. In some embodiments, the support device can be made from a disposable material (similar to paper or cloth). In other embodiments, the support device can be cleaned and reused.

Many fabrics known in the art may be used, depending on the desired characteristics such as elasticity, warmth, weight, breathability, stain resistance, absence of allergens, visual appeal and other factors. The present invention may be made of a single material or parts may be made of different materials. Flexible, non-fabric materials may also be used to provide special characteristics.

The disclosure further provides methods for preparing a support device for wearing during a MRI procedure, which include attaching a front panel having a bottom edge, a top edge, a left edge, a right edge, a font surface and a rear surface to a left arm sleeve having a bottom edge, a top edge, a front surface and a rear surface, the left arm sleeve extending from the left edge of the front panel; and attaching the front panel having the bottom edge, the top edge, the left edge, the right edge, the front surface and the rear surface to a right arm sleeve having a bottom edge, a top edge, a front surface and a rear surface, the right arm sleeve extending from the right edge of the front panel.

In some embodiments, the methods for preparing a support device include using an attachment means for attaching the left arm sleeve to the front panel; and using an attachment means for attaching the right arm sleeve to the front panel.

In other embodiments, the methods for preparing a support device include using an attachment means, wherein each attachment means further includes at least one zipper, at least one button, at least one snap, and/or at least one hook and loop fastener.

In some embodiments, the methods for preparing a support device include using an attachment means, wherein the attachment means for attaching the left arm sleeve to the front panel is a hook and loop fastener; and the attachment means for attaching the right arm sleeve to the front panel is a hook and loop fastener.

In other embodiments, the methods for preparing a support device include attaching at least one adjustable strap for securing the left arm sleeve and/or the right arm sleeve to the front panel of the device.

In some embodiments, the methods for preparing a support device include the support device of one or more pieces of material selected from one or more synthetic materials, one or more cotton materials, one or more cellulosic/viscose materials, one or more wool materials, one or more silk materials, one or more leather materials, one or more bast fiber materials, and/or one or more paper materials.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

What is claimed is:

1. A support device for wearing during a MRI procedure, comprising:
a front panel having a bottom edge, a top edge, a left edge, a right edge, a front surface and a rear surface;
a left arm sleeve having a bottom edge, a top edge, a front surface and a rear surface, the left arm sleeve extending from the left edge of the front panel; and
a right arm sleeve having a bottom edge, a top edge, a front surface and a rear surface, the right arm sleeve extending from the right edge of the front panel.

2. The support device of claim 1, further comprising:
an attachment means for attaching the left arm sleeve to the front panel; and
an attachment means for attaching the right arm sleeve to the front panel.

3. The support device of claim 2, wherein each attachment means further comprises at least one zipper, at least one button, at least one snap, and/or at least one hook and loop fastener.

4. The support device of claim 3, wherein the attachment means for attaching the left arm sleeve to the front panel is a hook and loop fastener, and the attachment means for attaching the right arm sleeve to the front panel is a hook and loop fastener.

5. The support device of claim 1, further comprising at least one adjustable strap for securing the left arm sleeve and/or the right arm sleeve to the front panel of the device.

6. The support device of claim 1, wherein the support device comprises one or more pieces of material selected from one or more synthetic materials, one or more cotton materials, one or more cellulosic/viscose materials, one or more wool materials, one or more silk materials, one or more leather materials, one or more bast fiber materials, and/or one or paper materials.

7. A method of preparing a support device for wearing during a MRI procedure, comprising:
attaching a front panel having a bottom edge, a top edge, a left edge, a right edge, a front surface and a rear surface to a left arm sleeve having a bottom edge, a top edge, a front surface and a rear surface, the left arm sleeve extending from the left edge of the front panel; and
attaching the front panel having the bottom edge, the top edge, the left edge, the right edge, the front surface and the rear surface to a right arm sleeve having a bottom edge, a top edge, a front surface and a rear surface, the right arm sleeve extending from the right edge of the front panel.

8. The method of preparing a support device of claim 7, further comprising:
using an attachment means for attaching the left arm sleeve to the front panel; and
using an attachment means for attaching the right arm sleeve to the front panel.

9. The method of preparing a support device of claim 8, wherein each attachment means further comprises at least one zipper, at least one button, at least one snap, and/or at least one hook and loop fastener.

10. The method of preparing a support device of claim 9, wherein the attachment means for attaching the left arm sleeve to the front panel is a hook and loop fastener, and the attachment means for attaching the right arm sleeve to the front panel is a hook and loop fastener.

11. The method of preparing a support device of claim 7, further comprising attaching at least one adjustable strap for securing the left arm sleeve and/or the right arm sleeve to the front panel of the device.

12. The method of preparing a support device of claim 7, wherein the support device comprises one or more pieces of material selected from one or more synthetic materials, one or more cotton materials, one or more cellulosic/viscose materials, one or more wool materials, one or more silk materials, one or more leather materials, one or more but fiber materials, and/or one or more paper materials.
